(19) United States
(12) Patent Application Publication
(10) Pub. No.: US 2025/0135236 A1
(43) Pub. Date: May 1, 2025

(54) CONFIGURABLE PHANTOM FOR END-TO-END VERIFICATION OF ADAPTIVE RADIOTHERAPY SYSTEMS

(71) Applicant: VANDERBILT UNIVERSITY, Nashville, TN (US)

(72) Inventor: Adam D. YOCK, Nashville, TN (US)

(21) Appl. No.: 18/936,386

(22) Filed: Nov. 4, 2024

Related U.S. Application Data
(60) Provisional application No. 63/545,951, filed on Oct. 27, 2023.

(51) Int. Cl. A61N 5/10

(52) U.S. Cl. CPC A61N 5/1075; A61N 5/103; A61N 2005/1076

(57) ABSTRACT

Systems and methods are provided for evaluating an adaptive radiotherapy system. An apparatus includes a housing formed from a first material having a first appearance under a selected imaging modality and a target that is inserted into the housing. The target includes a plurality of target components formed from a second material having a second appearance under the selected imaging modality. The plurality of target components are manipulable to assume at least a first configuration comprising a first subset of the plurality of target components and a second configuration comprising a second subset of the plurality of target components that is different from the first subset of the plurality of target components.

### RELATED APPLICATIONS

This application claims priority to U.S. Provisional Patent Application Ser. No. 63/546,951, filed Nov. 2, 2023 and entitled “CONFIGURABLE PHANTOM FOR END-TO-END VERIFICATION OF ADAPTIVE RADIOTHERAPY SYSTEMS,” which is hereby incorporated by reference in its entirety.

### TECHNICAL FIELD

This disclosure relates to medical treatment systems and is specifically directed to a configurable phantom for end-to-end verification of adaptive radiotherapy systems.

### BACKGROUND

Recent technological advances have facilitated the clinical implementation of online adaptive radiotherapy (online ART) whereby a new plan is created during an individual treatment fraction in response to a change in patient anatomy. To a large extent, the treatment planning process of online ART resembles the traditional offline process. However, the need for it to be completed quickly while the patient remains in position on the treatment machine introduces several important considerations. First, tasks that historically have been very time consuming, like contouring the tumor and normal tissues or optimizing a new fluence pattern, have been accelerated by fast computer hardware and intelligent algorithms. In addition, new demands are made of clinicians to conduct, review, and approve the results at the treatment console so as to ensure the same level of quality and safety as a conventional plan. Combining these new versions of familiar planning tasks into a novel, stream-lined, time-sensitive process has major implications on the staffing, workflow, and risk of the radiation delivered to patients.

Critical to establishing a safe and effective online ART program is for medical physicists to perform extensive commissioning tests upon installation of the technology and to establish a robust quality assurance program that repeats select tests periodically. Many components of the ART system can be tested individually through conventional methods. For example, the image quality of the on-board imaging system can be evaluated using conventional image quality phantoms and metrics, and the alignment of the imaging system and the treatment beam can be measured through Winston-Lutz-like tests. However, the primary novel characteristic of an online ART system is its full integration of each subsystem into a full treatment planning and delivery workflow. New methods and equipment are required to holistically validate this key functionality.

### SUMMARY

In one example, an apparatus includes a housing formed from a first material having a first appearance under a selected imaging modality and a target that is inserted into the housing. The target includes a plurality of target components formed from a second material having a second appearance under the selected imaging modality. The target is manipulable to assume at least a first configuration comprising a first subset of the plurality of target components and a second configuration comprising a second subset of the plurality of target components that is different from the first subset of the plurality of target components.

In another example, a method is provided for evaluating an adaptive radiotherapy system. A target is imaged in a first configuration, comprising a first set of at least two target components from a plurality of target components, to provide a first image. A morphology of the target is changed via one of removing a target component from the target or adding a target component from the plurality of target components to the target to provide a second configuration, comprising a second set of at least one target component from the plurality of target components. The target is imaged in the second configuration to provide a second image. A radiotherapy treatment plan is created for the adaptive radiotherapy system from one of the first image and the second image. An expected dose is determined for the radiotherapy treatment plan when applied to the target in one of the first configuration and the second configuration. The radiotherapy treatment plan is applied to the target in one of the first configuration and the second configuration to provide a dosage measurement at a dosimeter associated with the target. A metric representing a performance of the adaptive radiotherapy system is determined according to the expected dose and the dosage measurement.

In a further example, an apparatus includes a housing formed from a first material having a first appearance under a selected imaging modality and a target that is inserted into the housing. The target includes a plurality of target components formed from a second material having a second appearance under the selected imaging modality. The target is manipulable to assume at least a first configuration comprising a first subset of the plurality of target components and a second configuration comprising a second subset of the plurality of target components that is different from the first subset of the plurality of target components. The first configuration has a known geometric relationship to the second configuration representing a geometric transformation required to convert a morphology of the first configuration to a morphology of the second configuration.

### BRIEF DESCRIPTION OF THE DRAWINGS

FIG. 1 is a side view of an imaging device and a rigid phantom assembly;

FIG. 2 depicts one example of a configurable target for a phantom assembly for use in verifying an adaptive radiotherapy system;

FIG. 3 depicts an example of rigid phantom assembly for use in an adaptive radiotherapy system using configurable targets;

FIG. 4 illustrates examples of two treatment plans that can be implemented with a phantom assembly as disclosed herein; and

FIG. 5 depicts one method of evaluating an adaptive radiotherapy system using a configurable target.

### DETAILED DESCRIPTION

Online adaptive radiotherapy is an emerging technique that features the ability to rapidly re-plan the treatment at each session. Because the steps required in re-planning are both time-intensive and computationally-intensive, systems have been developed to make this technique technologically feasible. To verify the performance of these systems, users must conduct a battery of tests including a comprehensive end-to-end test of the entire process. A comprehensive end-to-end test is required to evaluate all steps and subsystems of the online ART system. These steps include (a) the creation of the original plan, (b) the imaging of a different anatomic configuration, (c) the potential creation of synthetic planning images, (d) the generation of contours and contour editing functionality, (e) the calculation of dose and the re-optimization of a new plan, and finally, (f) the delivery of radiation to be independently measured by a physical detector. Historically, there has been no straight forward solution that provides the medical physicist with a convenient tool to comprehensively evaluate an online ART system and ensure its integrity.

In order to address this observed deficiency, we have developed a phantom which is capable evaluating the entire online ART workflow from end-to-end. The verification process tests all the faculties and processes of the adaptive radiotherapy system including image acquisition and registration, structure re-contouring, plan reoptimization, dose calculation, plan quality assurance, and plan delivery independently, or in select or comprehensive combination. The phantom features a configurable radiotherapy treatment target that can be adapted via addition or removal of parts of the target to adjust the shape of the target among a plurality of shapes having known geometric relationships.

For the purpose of this application, a target component is added to a target when a target component formed from a target material is added to the target. It will be appreciated that this can include removal of a corresponding target component formed from a material other than the target material.

For the purpose of this application, a target component is removed from a target when a target component formed from a target material is removed from the target. It will be appreciated that this can include addition of a corresponding target component formed from a material other than the target material.

For the purpose of this application, a subset is used in the technical sense in which a subset can include all of the elements of the parent set. A subset comprising less than all of the elements of its parent set is referred to here as a proper subset.

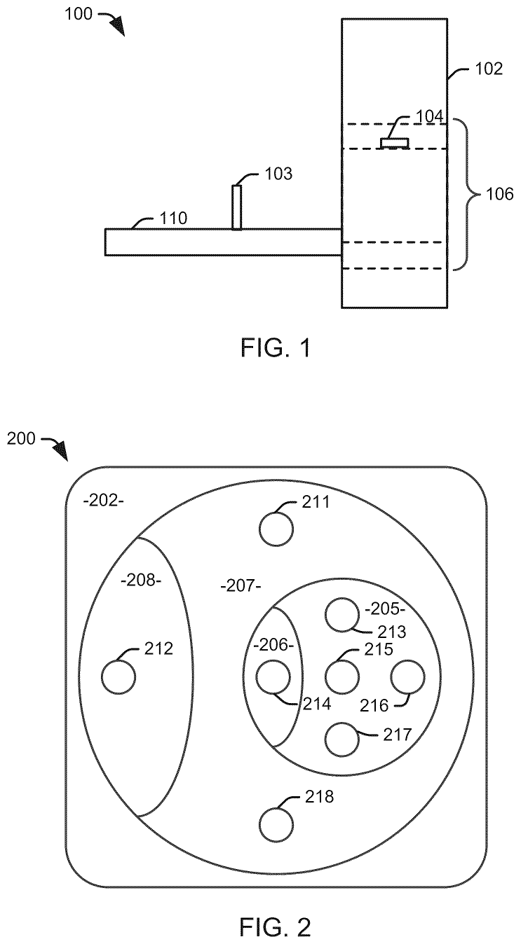

FIG. 1 illustrates a system 100 that includes a radiotherapy apparatus 102 and a phantom assembly 103. The radiotherapy apparatus includes a radiation source 104 which can provide radiation to a region of interest. In this example, the radiation source 104 is a radiotherapy treatment source for radiotherapy apparatus 100. The radiotherapy apparatus 102 also includes an imaging system 106, such as a computer tomography scanner, that can image subjects within the region of interest. It will be appreciated that the imaging system 106 can share components with the radiation source 104.

Generally, for radiotherapy treatment, the radiation source 104 emits a treatment beam, which is directed at and irradiates tissue of interest of a patient on a patient support 110, which is used to position a patient for treatment and imaging. The radiation source 104 can be rotated to one or more predetermined angular locations and/or the patient support 110 can be moved to facilitate directing the treatment beam according to a radiotherapy plan. As shown in FIG. 1, the phantom assembly 103 can be placed on the patient support 110 of the imaging device 106, and an image can be taken with the imaging device, allowing for the appearance of one or more targets on the phantom assembly 103. Since the size, shape, and orientation of the targets are known, the image captured at the imaging device 106 can be compared to an expected appearance of the target to evaluate the imaging device and the quality of images it produces. The rigid phantom assembly 103 can also be instrumented with various dosimeters and targeted with a treatment beam to measure the dose of ionizing radiation produced by the radiation source 104 at the target region of the phantom assembly.

In accordance with an aspect of the present invention, the phantom assembly 103 includes a housing and at least one target. The housing is formed from a first material having a desired appearance when imaged by the imaging device 106 and is configured to receive a target, which includes a plurality of target components formed from another material, generally selected to mimic a type of tissue. The target can have a plurality of configurations, with each of the plurality of configurations utilizing a unique subset of the plurality of components. The morphology of each configuration is precisely characterized and designed to have a known relationship to the morphology of the other targets, such as known differences in size, position, and shape, with the morphological relationships designed to mimic the ways in which a patient's anatomy can change throughout the course of treatment. The user can design a treatment plan based on one object and attempt to deliver it to another object featuring different morphology. A digital representation of the phantom can also be used for in-silico tests.

In one example, the phantom assembly 103 can be used to conduct end-to-end tests of a cone beam computed tomography (CBCT) guided online ART system. The process starts by first acquiring a computed tomography (CT) scan of the phantom 103 with the configurable target in a particular configuration. Then, a treatment plan is created based on this planning CT image. Finally, the plan is delivered to the phantom 103 one or more times while changing the phantom configurations. To verify the ability of the system to adapt the plan to the changing configurations, several geometric and dosimetric parameters can be compared between the original plan, the plan recalculated on the current CBCT, and the new plan re-optimized based on the current CBCT. In addition, to verify the accuracy of the delivered dose, physical measurements can be made in the phantom 103 and compared to an expected value.

FIG. 2 depicts one example of a configurable target 200 for a phantom assembly for use in verifying an adaptive radiotherapy system. In the illustrated example, the configurable target 200 includes a frame 202 configured to receive one or more of a plurality of target components 205-208. The frame 202 can be formed from a material that has an appearance for a given imaging modality that is similar to water. For example, when the imaging modality is X-ray computed tomography, the material of the frame 202 can be selected to have a linear attenuation value similar to that of water, such as RMI-457 Solid Water®, Virtual Water®, and Plastic Water®. In one example, the housing is formed from a high-density polyethylene slab.

Each of the plurality of target components 205-208 can be made from a material that has an appearance for a given imaging modality that is similar to a target tissue type. In one example, the target components 205-208 are formed from an acrylic and are intended to mimic the appearance of soft tissue in a computed tomography image. It will be appreciated, however, that a target could be formed from a high-density plastic that is intended to mimic the appearance of bone in a computed tomography image. In one example, the plurality of target components 205-208 can be made of different materials, which can mimic the appearance of one or more tissue types. In one example, the target components 205-208 can be selectively removed from and inserted into from the frame 202 to provide various configurations of the plurality of target components, each represented by a unique subset of the plurality of target components.

One or more of the plurality of target components 205-208 can include a hollow interior portion, accessible via one or more removable plugs 211-218, to allow for one or more dosimeters (e.g., thermoluminescent dosimeters (TLD), optically stimulated luminescent dosimeters (OSLD), ion chambers) to be inserted into the target area. In different implementations, the removable plugs and dosimeters could be located at various positions within each interior portion, including deliberately placed off-center to emphasize the varying shapes of targets. In some implementations, multiple dosimeters can be distributed through the targets, frames, or housing of the phantom. A set of phantom assemblies can be stacked to join the interior surfaces of the targets as to allow for a larger dosimeter to be placed within the stack of phantom assemblies. Alternatively or additionally, radiochromic films can be placed between phantom assemblies in a stack to measure a cross-sectional distribution of an applied dose of radiation. In practice, the phantom to be imaged by a given imaging system will have a desired thickness associated with the imaging modality.

In one example, the plurality of target components 205-208 can have a set of configurations with each configuration precisely designed to have a known geometric relationship relative to the other configurations. It will be appreciated that, for the purpose of this application, configurations are defined by the number of target components that are made from a material associated with the target, and the discussion below describes target components in these terms. It will be appreciated, however, that in practice, the target, when in a given configuration, can include other components, for example, having a same shape as other target components, that are made from a different material than the target material with a different appearance in the imaging modality. The known geometric relationship between configurations can be interpreted as the geometric transformation required to convert the morphology of an object represented by one configuration to the morphology of another configuration. The relationships can be selected to mimic changes in anatomy during radiotherapy. In the illustrated example, a first configuration can be generated with a first component 205 by itself, or, as discussed above, with the first component is formed from the target material and the other space within the target filled with components (e.g., components 206-208) that are formed from different material. A second configuration can be generated by inserting first and second components 205 and 206. In the illustrated implementation, the second configuration is substantially circular and the first configuration represents an asymmetric deformation of the second configuration. A third configuration can be generated by inserting first, second, and third components 205-207. A fourth configuration can be generated by inserting first, second, third, and fourth components 205-208. The fourth configuration is substantially circular and has a diameter substantially equal to twice that of the second configuration. The third configuration represents an asymmetric deformation of the fourth configuration. From these known relationships, it is possible to generate an expected dose for each object given a radiotherapy plan designed for another object. Specifically, it will be appreciated that these known relationships among the targets allow for simulation of the effects of changes in anatomy on dose calculations, allowing for appropriate evaluation of an adaptive radio therapy system.

Providing test objects of different morphologies can be achieved multiple ways. A target 200 can be shifted, rotated, or exchanged such that the object used for the original treatment plan is replaced by another. Alternatively, target components 205-208 can be adjusted to change the size or morphology of the target. For example, components 205-208 can be added, removed, moved relative to other components or rotated relative to other components to adjust the shape of the target. In one example, the third and fourth components 207 and 208 can be rotated relative to the frame 202 and the first and second components 205 and 206 can be rotated relative to the third and fourth components to effectively displace the first and second components off-axis. Accordingly, the phantom can be imaged and irradiated while simulating changes in tissue morphology, allowing for its use as a ground truth to evaluate adaptive radiotherapy workflows and processes.

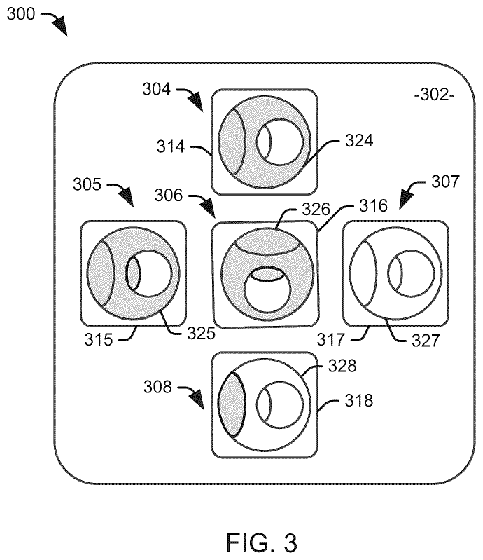

FIG. 3 depicts one example of a rigid phantom assembly 300 using configurable targets for use in verifying an adaptive radiotherapy system. The assembly 300 includes a housing 302 having a plurality of slots, each configured to receive one of a plurality of target inserts 304-308. The housing 302 can be formed from a material that has an appearance for a given imaging modality that is similar to water. For example, when the imaging modality is X-ray computed tomography, the material of the housing 302 can be selected to have a linear attenuation value similar to that of water, such as RMI-457 Solid Water®, Virtual Water®, and Plastic Water®. In one example, the housing is formed from a high-density polyethylene slab.

Each of the plurality of target inserts 304-308 can include a frame 314-318, made from a material similar to the housing 302 enclosing a configurable target 324-328, with each configurable target comprising a plurality of target components that are formed from a material that has an appearance for a given imaging modality that is similar to a target tissue type. It will be appreciated that each target 324-328 can have different target components to allow for a different set of configurations for that component or one or more targets 324-328 can share a set of target components, such that each target component in the plurality of target components associated with a first target (e.g., 324) has a shape congruent with a corresponding target component in the plurality of target components associated with a second target (e.g., 325). It will be appreciated, however, that the housing 302 can be configured to receive the configurable targets 324-328 directly, without the frames 314-318.

The target components for each configurable component can be selectively removed from and inserted into from the frame 314-318 to provide various configurations of the plurality of target components, each represented by a unique subset of the plurality of target components. In practice, “adding” a component to the target and “removing” a component from the target can include replacing a component formed from one material, for example, a material associated with the target, with a component formed from a second material, for example, a material associated with the housing, to effectively add or remove the component from the target configuration.

In one example, the targets 324-328 are formed from an acrylic and are intended to mimic the appearance of soft tissue in a computed tomography image. It will be appreciated, however, that a target could be formed from a high-density plastic that is intended to mimic the appearance of bone in a computed tomography image. In one example, one or more targets can be made of different materials, which can mimic the appearance of one or more tissue types. In one example, the inserts 304-308 can be removed from the housing 302 and either replaced with another insert, either one of the depicted inserts 304-308 or another insert, (not shown) or rotated ninety, one hundred eighty, or two hundred seventy degrees. In another example, the inserts 304-308 can be octagonal to allow rotation to be performed in forty-five degree increments. In a further example, the inserts can be circular to allow for arbitrary rotation. It will be appreciated that various shapes, generally regular polygons to allow for various rotations of the inserts.

One or more target components for each target 324-328 can include a hollow interior portion, accessible via one or more removable plugs, to allow for one or more dosimeters (e.g., thermoluminescent dosimeters (TLD), optically stimulated luminescent dosimeters (OSLD), ion chambers) to be inserted into the target area. In different implementations, the removable plugs and dosimeters could be located at various positions within each interior portion, including deliberately placed off-center to emphasize the varying shapes of targets. In some implementations, multiple dosimeters can be distributed through the targets, frames, or housing of the phantom. A set of phantom assemblies can be stacked to join the interior surfaces of the targets as to allow for a larger dosimeter to be placed within the stack of phantom assemblies. Alternatively or additionally, radiochromic films can be placed between phantom assemblies in a stack to measure a cross-sectional distribution of an applied dose of radiation. In practice, the phantom to be imaged by a given imaging system will have a desired thickness associated with the imaging modality. A phantom of the appropriate thickness can be formed from a single phantom assembly 300 or multiple, thinner phantom assemblies that are stacked to provide the desired thickness.

In one example, a set of the configurable targets 324-328 can be configured, via removal or insertion of their constituent components to have a known geometric relationship relative to the other targets. In FIG. 3, removed and replaced components are shaded in gray. The known geometric relationship between objects can be interpreted as the geometric transformation required to convert the morphology of one object to that of the other. The relationships can be selected to mimic changes in anatomy during radiotherapy. In the illustrated example, a first target 324 is configured in a first configuration that is substantially circular, a second target 325 is configured in a second configuration representing an asymmetric deformation of the first configuration, a third target 326 is configured in the second configuration, but rotated ninety degrees relative to the second target, a fourth target 327 is configured in a third configuration that is substantially circular with an area about four times that of the first target, and the fifth target 328 is configured in a fourth configuration that represents an asymmetric deformation of the third configuration. It will be appreciated that these known relationships among the targets allow for simulation of the effects of changes in anatomy on dose calculations, allowing for appropriate evaluation of an adaptive radiotherapy system.

Changing test objects of different morphologies can be achieved multiple ways. A given insert 304-308 of the phantom 300, or the entire phantom, can be shifted, rotated, or exchanged such that the object used for the original treatment plan is replaced by another. For example, rotating the housing 302 of the phantom replaces one test object, represented by a target 324-328, with another, allowing for evaluation of a deformable image registration (DIR) system with respect to changes in position, orientation, and shape depending on the geometric relationship of the two objects. Alternatively, components comprising the target can be adjusted, for example, rotated, exchanged, added, or removed, to change the configuration of the target, and accordingly, the tissue morphology represented by the target. Accordingly, the phantom can be imaged and irradiated while simulating changes in tissue morphology, allowing for its use as a ground truth to evaluate adaptive radiotherapy workflows and processes.

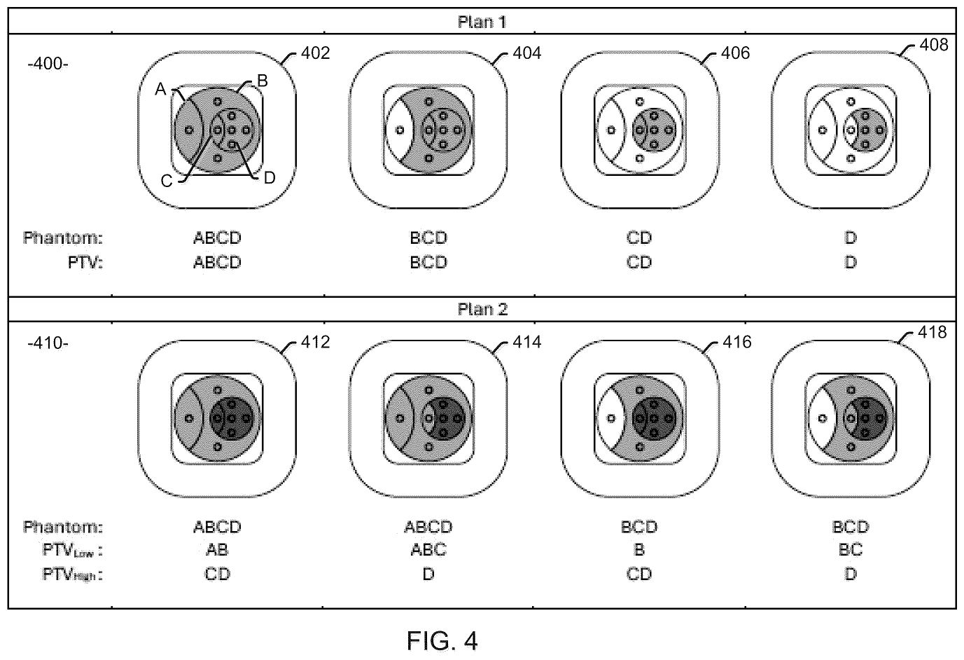

In one example, illustrated as FIG. 4, a first treatment plan 400 can include four configurations, each providing a different planning target volumes (PTV). In this example, a radiotherapy target structure features an outer body, frame, and a configurable insert capable of representing multiple target morphologies. When viewed in profile as an axial slice through the phantom, the configurable insert comprises two nested, non-concentric circles, each composed of a lens-shaped piece (labeled A and C) and a crescent moon-shaped piece (labeled B and D). Each piece features one or more channels capable of receiving a variety of radiation detectors that can also be plugged when not in use.

In the first treatment plan 400, representing, for example, a prescription dose level, the body, frame, and one set of configurable insert components were all made out of high-density polyethylene (HDPE) with CT Hounsfield Units around −60 HU. Components made from this material are shown as white throughout FIG. 4. Additional components for the configurable insert were made out of acrylic, with CT Hounsfield Units around 120 HU. Components made from this material are shown as light grey throughout FIG. 4. By assembling the configurable insert using different combinations of the components made from the two materials, multiple related target morphologies can be achieved. For example, starting from an acrylic target configuration 402 composed of A, B, C, and D (i.e., the ABCD configuration), replacing the acrylic A with the HDPE version has the effect of changing the radiographic appearance of the target from the large circle to the large crescent configuration 404 (BCD). Additionally swapping the acrylic B and subsequently acrylic C for their HDPE counterparts further changes the target to the small circle 406 (CD) and small crescent 408 (D), respectively. For any particular target configuration, the large and small circles of the insert remain free to rotate. As a result, the user can achieve additional geometries by introducing certain rotations and/or translations of the targets and detector channels. In total, the user therefore has control over the size, shape, and/or position of the target simply by adjusting the pieces of the configurable insert. Lastly, as the insert components are analytical shapes, their boundaries and volumes are known precisely such that changes in target morphology (size, shape, and position) can be determined and used as a ground truth.

In a second treatment plan 410, a given target can be configured to provide two separate planning target volumes (PTVs), for example, to receive two separate doses. Each PTV can be formed from a material having different properties under the selected imaging modality, or both volumes can be made from a similar material, such as acrylic. For example, in addition to the sets of target components made from HDPE and acrylic, an additional set of components can be included that are made from a solid surface material (e.g., Corian®) that is intended to mimic the appearance of bone in a computed tomography image. In the second treatment plan, target components that are part of a low-dose target are shaded light gray and target components that are part of a high-dose target are shaded dark grey regardless of the material from which they are made. In a first configuration 412, the smaller of the non-concentric circles, comprising components C and D, provides a high dose target, and the remainder of the phantom, comprising components A and B, provide a low dose target. In the second configuration 414, component C is included in the low dose target comprising components A, B, and C, with a high dose target comprising only component D. In the third configuration 416, component A is replaced with its HDPE counterpart to be removed from any target and component C is returned to the high dose target to provide a low dose target comprising only component B, and a high dose target comprising components C and D. In the fourth configuration, component C is once again included in the low dose target comprising components B and C, with a high dose target comprising only component D.

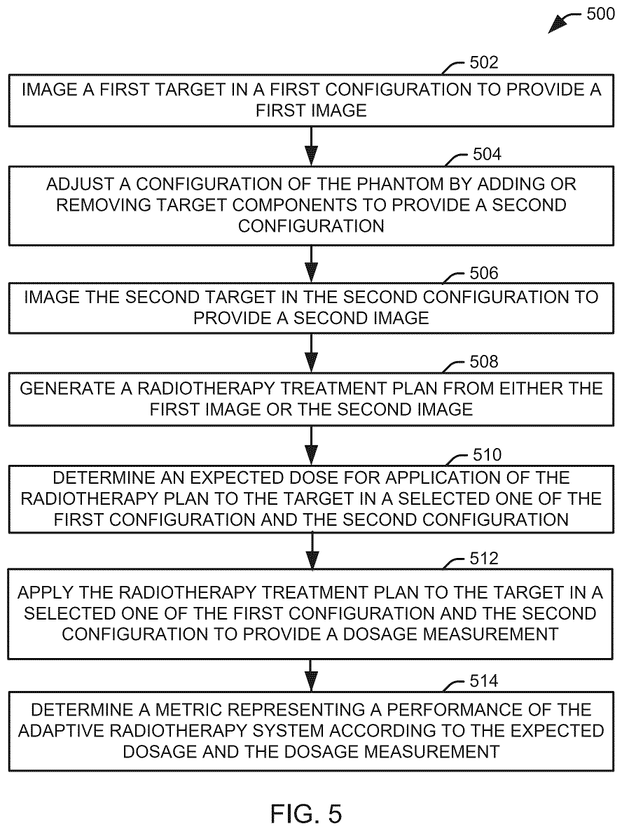

In view of the foregoing structural and functional features described above in FIGS. 1-4, an example method will be better appreciated with reference to FIG. 5. While, for purposes of simplicity of explanation, the method of FIG. 5 is shown and described as executing serially, it is to be understood and appreciated that the present invention is not limited by the illustrated order, as some actions could in other examples occur in different orders and/or concurrently from that shown and described herein.

FIG. 5 illustrates one example of a method 500 for evaluating an adaptive radiotherapy system using a configurable target. At 502, a target in a first configuration, comprising a first set of at least one target component from a plurality of target components, is imaged to provide a first image. It will be appreciated that the target can be imaged at an adaptive radiotherapy system or a separate imaging system, such as a computed tomography (CT) scanner or a magnetic resonance imaging (MRI) system. At 504, a morphology of the target is changed by either removing a target component from the target or adding a target component to the target to provide a second configuration comprising a second set of at least two target components of the plurality of target components. It will be appreciated that a target component can be added or removed as part of an exchange of a target component with another target component. At 506, the second target is imaged in the second configuration to provide a second image. At 508, a radiotherapy treatment plan is created for the adaptive radiotherapy system from either the first image or the second image. At 510, an expected dose for the radiotherapy plan for the target in a selected one of the first configuration and the second configuration is determined. At 512, the radiotherapy treatment plan is applied to the target in one of the first configuration and the second configuration to provide a dosage measurement at a dosimeter associated with the target. A metric representing the performance of the radiotherapy system is determined according to the expected dose and the dosage measurement at 514. It will be appreciated that the first configuration has a known geometric relationship with the second configuration, and the metric representing the performance of the adaptive radiotherapy system can take into account the known geometric relationship between the first configuration and the second configuration in addition to the expected dose and the dosage measurement.

It will be appreciated that, in the target of FIG. 2, the geometric relationships among the various target configurations are known a priori, and thus multiple comparisons can be made among expected doses for the two treatments plans, as applied to target in either configuration, or any measured dosages themselves, to either refine the performance metric or to generate an additional performance metric. For example, expected doses could be calculated for the application of the radiotherapy treatment plan to each of the first or second targets or the application of a second radiotherapy treatment plan, generated from the other of the first image and the second image to the target in either of the first or second configurations, and corresponding dosage measurements can be made and compared to these expected doses or to one another as part the evaluation of the radiotherapy system.

Where multiple metrics are used, each metric can represent a performance of a different stage or set of stages of an adaptive radiotherapy system. For example, a comparison of the expected dosage for a first radiotherapy plan as applied to the first target configuration to the expected dosage for a second radiotherapy plan as applied to the second target configuration can be used to evaluate the plan reoptimization stage of the adaptive radiotherapy system. Similarly, comparing the measured dosage for the first radiotherapy plan as applied to the second target configuration to the measured dosage for the second radiotherapy plan as applied to the second target configuration can be used to evaluate the overall effectiveness of the adaptive radiotherapy system in improving the outcome given the change in anatomy represented by the second target configuration. Accordingly, the performance of the adaptive radiotherapy system can be evaluated both overall as well as at the level of the individual subsystems.

What have been described above are examples of the invention. It is, of course, not possible to describe every conceivable combination of components or methodologies, but one of ordinary skill in the art will recognize that many further combinations and permutations of the invention are possible. Accordingly, the invention is intended to embrace all such alterations, modifications and variations that fall within the scope of the appended claims and the application. Additionally, where the disclosure or claims recite “a,” “an,” “a first,” or “another” element, or the equivalent thereof, it should be interpreted to include one or more than one such element, neither requiring nor excluding two or more such elements. As used herein, the term “includes” means includes but not limited to, the term “including” means including but not limited to. The term “based on” means based at least in part on.

What is claimed is:

1. An apparatus comprising:
a housing formed from a first material having a first appearance under a selected imaging modality; and
a target that is inserted into the housing, the target comprising a plurality of target components formed from a second material having a second appearance under the selected imaging modality and manipulable to assume at least a first configuration comprising a first subset of the plurality of target components and a second configuration comprising a second subset of the plurality of target components that is different from the first subset of the plurality of target components.

2. The apparatus of claim 1, the first configuration having a known geometric relationship to the second configuration.

3. The assembly of claim 2, the known geometric relationship comprising a geometric transformation required to convert a morphology of the first configuration to the morphology of the second configuration.

4. The assembly of claim 3, wherein the first configuration represents the second configuration with an asymmetric deformation.

5. The assembly of claim 3, wherein the first configuration represents the second configuration with a known change in volume.

6. The assembly of claim 1, wherein the first subset of the plurality of target components comprises all of the target components.

7. The assembly of claim 1, the target further comprising a frame that is inserted into the housing to receive a subset of the plurality of target components, the frame being removable from the housing.

8. The assembly of claim 7, the housing being configured to receive the frame in at least two orientations, such that the target can be rotated relative to the housing.

9. The assembly of claim 1, where the target is a first target, the plurality of target components is a first plurality of target components, and the apparatus further comprises a second target that is inserted into the housing, the second target comprising a second plurality of target components and manipulable to assume at least a third configuration comprising a first subset of the second plurality of target components and a fourth configuration comprising a second subset of the second plurality of target components that is different from the first subset of the second plurality of target components.

10. The assembly of claim 9, wherein each of the first plurality of target components has a shape congruent with a corresponding one of the second plurality of target components.

11. A method for evaluating an adaptive radiotherapy system, the method comprising:
imaging a target in a first configuration, comprising a first set of at least two target components from a plurality of target components, to provide a first image;
changing a morphology of the target via one of removing a target component from the target or adding a target component from the plurality of target components to the target to provide a second configuration, comprising a second set of at least one target component from the plurality of target components;
imaging the target in the second configuration to provide a second image;
creating a radiotherapy treatment plan for the adaptive radiotherapy system from one of the first image and the second image;
determining an expected dose for the radiotherapy treatment plan when applied to the target in one of the first configuration and the second configuration;
applying the radiotherapy treatment plan to the target in one of the first configuration and the second configuration to provide a dosage measurement at a dosimeter associated with the target; and
determining a metric representing a performance of the adaptive radiotherapy system according to the expected dose and the dosage measurement.

12. The method of claim 11, wherein the first configuration has a known geometric relationship with the second configuration, and determining the metric representing the performance of the adaptive radiotherapy system comprises determining the metric representing the performance of the adaptive radiotherapy system according to the expected dose, and the dosage measurement, and the known geometric relationship between the first configuration and the second configuration.

13. The method of claim 11, wherein creating the radiotherapy treatment plan comprises creating the radiotherapy treatment plan from the second image and determining the expected dose for the radiotherapy treatment plan when applied to the one of the first target and the second target comprises determining the expected dose for the radiotherapy treatment plan when applied to the second target.

14. The method of claim 13, wherein the dosage measurement is a first dosage measurement, and the radiotherapy plan is a first radiotherapy plan, the method further comprising:
creating a second radiotherapy treatment plan for the adaptive radiotherapy system from the first image; and
applying the second radiotherapy treatment plan to the second target on the phantom to provide a second dosage measurement at the dosimeter associated with the phantom;
wherein evaluating a performance of the adaptive radiotherapy system comprises evaluating the performance of the adaptive radiotherapy system according to the expected dose, the first dosage measurement and the second dosage measurement.

15. The method of claim 14, wherein the metric is a first metric and evaluating the performance of the adaptive radiotherapy system according to the expected dose, the first dosage measurement and the second dosage measurement comprises comparing the first dosage measurement and the second dosage measurement to generate a second metric.

16. An apparatus comprising:
a housing formed from a first material having a first appearance under a selected imaging modality; and
a target that is inserted into the housing, the target comprising a plurality of target components formed from a second material having a second appearance under the selected imaging modality and manipulable to assume at least a first configuration comprising a first subset of the plurality of target components and a second configuration comprising a second subset of the plurality of target components that is different from the first subset of the plurality of target components, wherein the first configuration has a known geometric relationship to the second configuration representing a geometric transformation required to convert a morphology of the first configuration to a morphology of the second configuration.

17. The apparatus of claim 16, the target further comprising a frame that is inserted into the housing to receive a subset of the plurality of target components, the housing being configured to receive the frame in at least two orientations, such that the target can be rotated relative to the housing.

18. The assembly of claim 16, where the target is a first target, the plurality of target components is a first plurality of target components, and the apparatus further comprises a second target that is inserted into the housing, the second target comprising a second plurality of target components and manipulable to assume at least a third configuration comprising a first subset of the second plurality of target components and a fourth configuration comprising a second subset of the second plurality of target components that is different from the first subset of the second plurality of target components, each of the first plurality of target components having a shape congruent with a corresponding one of the second plurality of target components.

19. The assembly of claim 16, wherein the first configuration represents the second configuration with one of an asymmetric deformation and a known change in volume.

20. The assembly of claim 16, wherein the first subset of the plurality of target components comprises all of the target components, and the second subset of the plurality of target components is a proper subset of the plurality of target components.
